(19) United States
(12) Patent Application Publication
(10) Pub. No.: US 2011/0295025 A1
(43) Pub. Date: Dec. 1, 2011

(54) PROCESS FOR THE PREPARATION OF CHOLYL-L-LYSINE

(76) Inventors: Tomos Huw Roberts, Bangor (GB); Dafydd Thomas, Holywell (GB)

(21) Appl. No.: 13/201,266

(22) PCT Filed: Feb. 10, 2010
(86) PCT No.: PCT/GB10/50205

§ 371 Date: Aug. 12, 2011

(30) Foreign Application Priority Data
Feb. 12, 2009 (GB) 0902287.2

(51) Int. Cl. C07J 75/00

(52) U.S. Cl. 552/554

(57) ABSTRACT

A process for the preparation of cholyl-L-lysine comprising the steps of: —(a) reacting N-ε-CBZ-cholyl-L-lysine with a hydrogen source in the presence of a catalyst in a solvent comprising one or more alkanols; (b) removing the catalyst; (c) optionally diluting the resulting reaction mixture with water and optionally adjusting the pH of the resultant reaction mixture to a pH less than or equal to about 4; (d) removing the bulk of the alkanol whilst ensuring that the alkanol content of the resultant mixture is maintained above about 3% w/w of the remaining mixture; (e) extracting the resultant mixture from step (d) with an organic solvent; (f) adjusting the pH of the aqueous layer to a pH of about 4.5 or greater to precipitate cholyl-L-lysine; (g) isolate the precipitate.

The present invention relates to a process for the preparation of cholyl-L-lysine.

Cholyl-L-lysine (6-amino-2-((4R)-4-((3R,5S,7R,8R,9S,10S,12S,13R,17R)-3,7,12-trihydroxy-10,13-dimethylhexadecahydro-1H-cyclopenta[a]phenanthren-17-yl) pentanamido)hexanoic acid) is a key intermediate in the synthesis of fluorescein lisicol (previously known as cholyl-lysylfluorescein or CLF; N6-({3′,6′-dihydroxy-3-oxospiro[isobenzofuran-1(3H), 9′-xanthen]-5-yl}thiocarbamoyl)-N2-(3,7,12-trihydroxy-5-cholan-24-oyl)-L-lysine). The use of fluorescent bile acid derivatives, and CLF in particular, in a method for the determination of the liver function of a human or animal subject is described in EP1,003,458 (Norgine Europe BV).

C. O. Mills et al Biochimica & Biophysica Acta 1115 (1991) 151-156, describes the synthesis of cholyl-L-lysine by transfer hydrogenation of N-ε-CBZ-cholyl-L-lysine (6-(benzyloxycarbonylamino)-2-((4R)-4-((3R,5S,7R,8R,9S,10S,12S,13R,17R)-3,7,12-trihydroxy-10,13-dimethylhexadecahydro-1H-cyclopenta[a]phenanthren-17-yl)pentanamido)hexanoic acid). However, the workup procedure described by Mills et al requires the crude reaction product to be treated with base (NaOH) and then acid (HCl) followed by chromatographic purification of the crude reaction mixture in order to obtain the desired product free from formate salt. Such a workup procedure is both expensive and becomes unworkable and uneconomic when the reaction is scaled up to produce kilo quantities of this intermediate.

A synthesis of cholyl-L-lysine is also described in WO02/12267 (Norgine Europe BV). An extractive workup procedure was used in this case, whereby the methanolic reaction mixture was poured into approximately 7 volumes of water. The resulting acidic aqueous phase was then extracted twice with approximately 2 volumes of ethyl acetate to remove organic impurities. The resultant aqueous solution was adjusted to pH4.5-5.0 and cooled to precipitate the desired product. In the example given, 190 g of starting N-ε-CBZ-cholyl-L-lysine required the use of over 22 litres of solvent/water in the workup procedure.

In summary, neither of the prior art processes is unworkable on scale up and there is a long-felt need in the industry to provide a process which can be used safely and economically to produce kilo or multi-kilo batches of cholyl-L-lysine and which does not require protracted reaction times nor elaborate workup procedures.

A main objective of the present invention is to provide a high yielding process for the preparation of cholyl-L-lysine.

Another object of the present invention is to provide a process for the preparation of cholyl-L-lysine with relatively short reaction times.

Yet another object of the present invention is to provide a process for the preparation of cholyl-L-lysine in substantially pure form without elaborate workup procedures and without the need for chromatographic purification.

The present invention may be described by the following reaction scheme: —

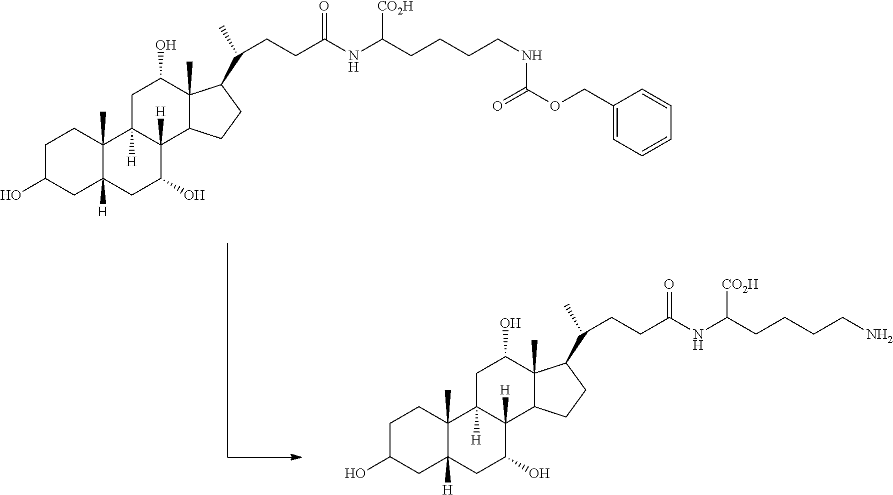

Accordingly there is provided a process for the preparation of cholyl-L-lysine comprising the steps of: —
(a) reacting N-ε-CBZ-cholyl-L-lysine with a hydrogen source in the presence of a catalyst in a solvent comprising one or more alkanols;
(b) removing the catalyst;
(c) optionally diluting the resulting reaction mixture with water and optionally adjusting the pH of the resultant reaction mixture to a pH less than or equal to about 4;
(d) removing the bulk of the alkanol whilst ensuring that the alkanol content of the resultant mixture is maintained above about 3% w/w of the remaining mixture;
(e) extracting the resultant mixture from step (d) with an organic solvent;
(f) adjusting the pH of the aqueous layer to a pH of about 4.5 or greater to precipitate cholyl-L-lysine;
(g) isolating the precipitate.

We have unexpectedly found that ensuring that some of the alkanol is retained in the resultant mixture in step (d) unexpectedly prevents unwanted precipitation during the aqueous extractive workup that follows.

Preferred alkanols in step (a) are C₁ to C₅ straight chain or branched chain alkanols.

Preferably the alkanol used as a solvent in step (a) is methanol.

In a further preferred embodiment the solvent in step (a) is a mixture of ethanol and methanol. Ethanol is typically used to moisten the catalyst at the start of the hydrogenation step.

Preferably the ratio of methanol to ethanol is in the range 5 to 1 v/v to 20:1 v/v and more preferably the ratio of methanol to ethanol is in the order of 10:1 v/v.

Where the methanol content of the resultant mixture after removal of most of the alkanol drops below about 3% w/w, there is a tendency for a precipitate to form. The nature of this precipitate, which tends to be particularly fine, makes it very difficult and time-consuming to filter off. If left, it obscures the interface between the aqueous layer and the solvent layer.

Preferably the alkanol content of the resultant mixture in step (d) is maintained at or above about 5% w/w of the remaining mixture. A preferred alkanol content of the mixture after partial removal of the alkanol is between 3% to 12% w/w and a particularly preferred alkanol content is between 5% to 10% w/w. For example, preferably the methanol content of the resultant mixture in step (d) is maintained at or above about 5% w/w of the remaining mixture. Where the solvent in step (a) is a mixture of ethanol and methanol, preferably the methanol content of the resultant mixture in step (d) is maintained at or above about 5% w/w of the remaining mixture.

Preferably the alkanol content of the resultant mixture in step (d) is maintained in the range of about 5-10% w/w of the remaining mixture. About 7% alkanol w/w is a particularly preferred concentration. For example, preferably the methanol content of the resultant mixture in step (d) is maintained in the range of about 5-10% w/w of the remaining mixture.

Partial removal, as opposed to substantially complete removal, of the alkanol from the reaction mixture following hydrogenation results in several advantages. Not only does it prevent or substantially reduce precipitation, it also enables much smaller volumes of water and organic solvent to be used in the extractive procedure than would otherwise be possible.

Preferably the process in step (a) is a transfer hydrogenation process. Transfer hydrogenation is considered safer than the use of hydrogen gas.

Preferably the transfer hydrogenation process utilizes formic acid in methanol.

Preferably the transfer hydrogenation process in step (a) is carried out at about 25° C. to 45° C., and more preferably the transfer hydrogenation process in step (a) is carried out at about 30° C. to 45° C., with a particularly preferred temperature range being 35° C. to 45° C. These temperature ranges give short reaction times, in the order of 3 to 4 hours, and also encourage the reaction to go to completion without requiring additional catalyst.

In the event that the transfer hydrogenation process in step (a) is incomplete, the catalyst is removed and fresh catalyst is added until complete conversion is observed, re-dosing with catalyst as necessary.

Preferably the organic solvent used in the extraction process in step (e) is ethyl acetate.

In relation to the reaction and workup conditions used in the hydrogenation reaction, a key feature involves an evaporative workup to remove most of the alkanol, whilst maintaining a minimum concentration of alkanol in the resultant mixture after the transfer hydrogenation reaction and during evaporative workup. In the example given below the alkanol used is methanol. During the removal of methanol the temperature is maintained below about 50° C. to ensue there is no thermal degradation of the product. The evaporation is limited to 2 hours and an in-process check at this time confirms that the methanol content of the resultant solution is between 5 and 10% by weight.

During the transfer hydrogenation workup procedure the methanolic reaction mixture is diluted with water and the methanol removed under reduced pressure to give an aqueous solution containing product that is extracted with ethyl acetate to remove organic impurities. However, we have observed that there was formation of a large amount of precipitate during the ethyl acetate extraction separation stage of the workup. This precipitate leads to a prolonged workup, due to the solid interfering with the separation of the organic and aqueous phases. In order to achieve separation of the phases the solid was removed by filtration and the workup continued. Due to the slow nature of the filtration, the formation of the precipitate added significantly to the time taken for the workup as well as potentially having an adverse effect on the yield of the product.

It was unexpectedly found that, by leaving a residual amount of methanol present in the mixture after evaporation, the reaction workup proceeded much more quickly and with less solid deposited at the water/ethyl acetate interface than was observed during workup of reactions where substantially all the methanol was removed. Normal evaporative workup resulted in a methanol content of the resultant mixture in the region of 0.02% w/w to 0.2% w/w.

Trial reactions were performed and these indicated that a methanol concentration after evaporative workup of greater than about 3% w/w resulted in a significant decrease in precipitate formation during the aqueous/ethyl acetate extractions. More preferably the methanol concentration is preferably greater than about 5% w/w. Concentrations of methanol in the order of about 5% to 10% w/w are acceptable and about 7% methanol w/w is a particularly preferred concentration.

It will be understood that if, after evaporation, the methanol concentration is less than the desired value, additional methanol can be added until the desired w/w % concentration is achieved.

It will also be appreciated that ethyl acetate is only one of a range of organic solvents that could be used in this extractive workup. For example, dichloromethane could be used and this has the advantage that the desired product is in the upper, aqueous layer, simplifying the extraction process.

The reaction conditions under which the transfer hydrogenation process is carried out have a significant bearing on the time taken for the reaction to go to completion. In particular the reaction temperature is an important factor.

Hydrogenation reactions carried out within the temperature range 50-60° C. were observed to stall before completion. This would result in poor isolated yield of product due to incomplete conversion of the starting material. It was noted that re-dosing these reactions with catalyst and formic acid progressed the reaction. However re-dosing with catalyst alone did not progress the reaction. This suggested that the formic acid may be consumed at higher temperature without taking part in the hydrogenation of the starting material.

Investigation into lowering the temperature range for the reaction, to 30-40° C. resulted in complete reaction being observed in less than 3 hr, determined by complete consumption of the starting material. The scale of the reaction was increased and a reaction based on 50 g of starting material carried out. This reaction was monitored closely over an initial 3 hr period to follow the reaction progress, and the results are shown in Table 1. It can be seen the reaction was complete within 1.5-2.0 hr. (using UV detection).

| | Starting | |
|---|---|---|
| Sample | Material % | Product % |
| 0.5 hr | 50 | 50 |
| 1.0 hr | 16 | 84 |
| 1.5 hr | 2 | 98 |
| 2.0 hr | 1 | 99 |

Table 1: process analytical data showing progress of 50 g scale reaction.

The cholyl-L-lysine prepared according to the present invention has a number of uses. For example, it can be used with fluorescein-5-isothiocyanate (FITC; 3′,6′-dihydroxy-5-isothiocyanato-3H-spiro[isobenzofuran-1,9′-xanthen]-3-one) to synthesise fluorescein lisicol (previously known as cholyl-lysylfluorescein or CLF) according to Scheme 1 below:

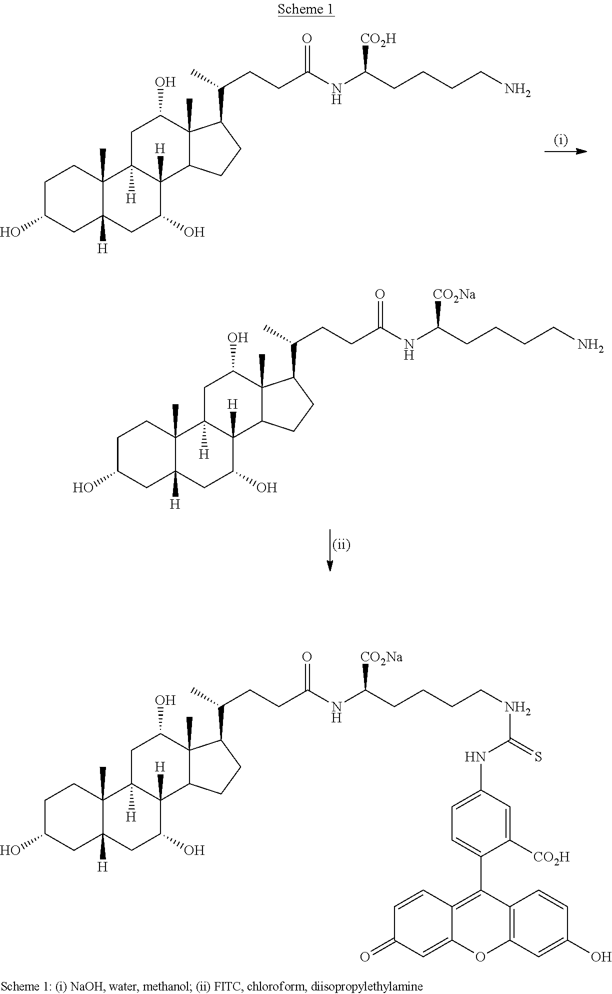

### EXAMPLE 1

### Preparation of N-ε-CBZ-Cholyl-L-Lysine

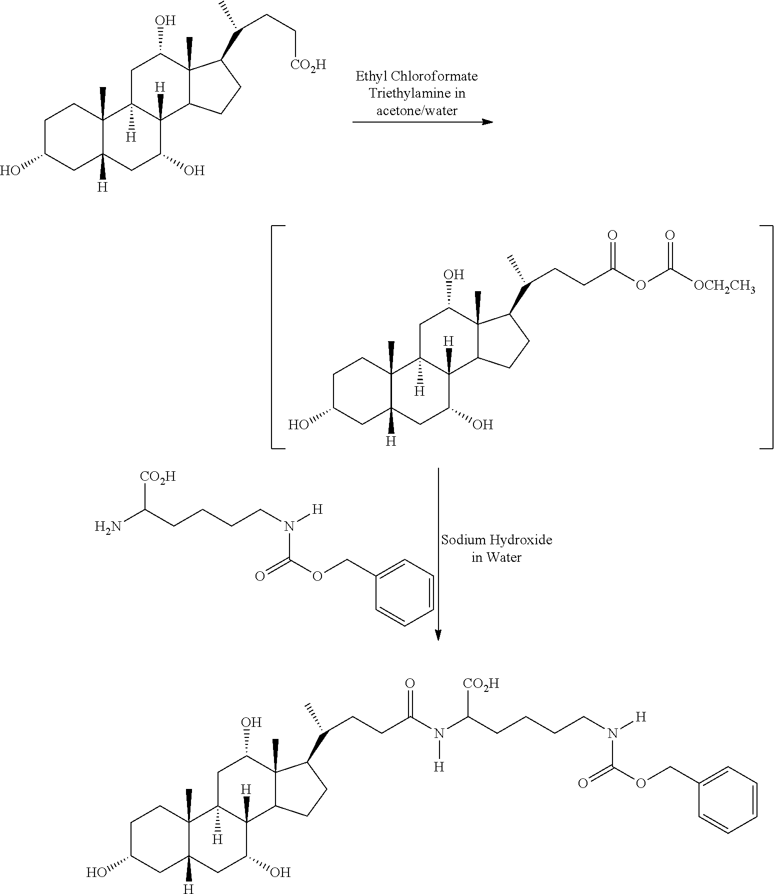

### Reaction Vessel 1

Acetone (1.1 L) was charged to a 5 L flask and stirring was commenced. Cholic acid (146 g) was charged to the solution at ambient temperature followed by triethylamine (70 ml). After 30 minutes, the contents of the flask were cooled to 5-15° C. and ethyl chloroformate (34 ml) was added dropwise over a 20 minute period with the internal temperature controlled at 20 to 25° C. The solution was stirred for a further 85-95 minutes.

### Reaction Vessel 2

Water (800 ml) was charged to a 2 L flask and stirring was commenced. Sodium hydroxide (15 g) was charged to the flask and the reaction was stirred for 10 minutes. N-ε-CBZ-L-Lysine (100 g) was charged to the vessel and the reaction was stirred for a further 60 minutes.

The contents of vessel 2 were charged, as rapidly as possible, to vessel 1 followed by stirring for a further 2-4 hours. The pH of the reaction mixture was adjusted to pH 2.0 to 2.5 using 1M Hydrochloric acid, maintaining the temperature below 30° C. by controlling the addition rate. The contents of vessel 1 were transferred to a suitable separating funnel, ethyl acetate (1.0 L) was charged and the solution was vigorously agitated for 5 minutes. The two layers were separated and the lower aqueous layer was recharged to the separating funnel. Ethyl acetate (1.0 L) was charged and the extraction procedure was repeated.

The ethyl acetate layers were combined and recharged to the separating funnel. Water (5.0 L) was charged to the separating funnel and the reaction mixture was vigorously stirred for 5 minutes. The two layers were separated and the upper organic layer was separated and dried with sodium sulphate. The solution was filtered, the filter pad washed with ethyl acetate (1.0 L), and the solvent removed on a rotary evaporator (temp 35 to 50° C.) to afford N-ε-CBZ-cholyl-L-lysine as a white solid.

m/z=371.6 (M+1)

m.p=110-114° C.

TLC (silica gel 60) Rf=0.15 (20% Methanol/dichloromethane)

IR (KBr) 3340, 2922, 2853, 1703, 1537, 1460, 1376, 1252 cm⁻¹

### EXAMPLE 2

### Preparation of Cholyl-L-Lysine

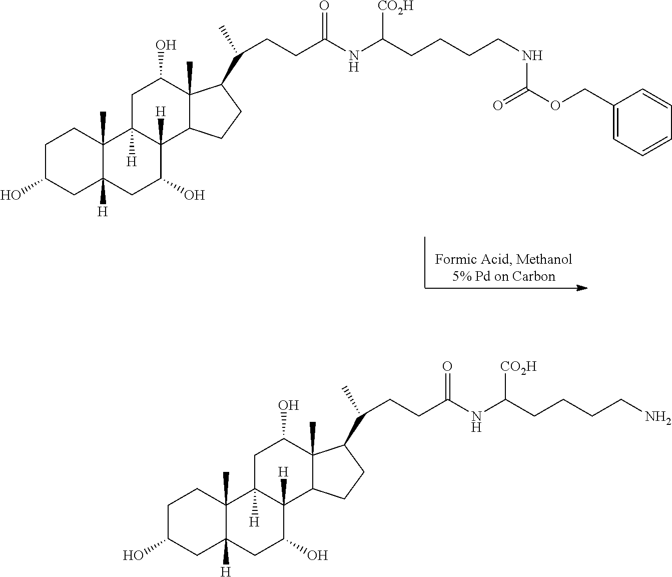

5% palladium on carbon catalyst (23 g) was charged to a 5 L flask at ambient temperature. Ethanol (230 ml) was slowly charged to the vessel. N-ε-CBZ-cholyl-L-lysine (0.23 Kg) was contained in a 6 L evaporation flask, methanol (2.3 L) was charged to the 6 L evaporation flask and stirring was commenced to dissolve the starting material. The contents of the 6 L flask were transferred to the 5 L flask at ambient temperature and stirring was continued. Formic acid (0.35 L) was charged to the vessel in one portion followed by a 5 to 15 minute subsurface nitrogen purge. The reaction contents were heated to an internal temperature of 35 to 45° C. and stirring continued for 3-3.5 hours and the reaction sampled for in-process analysis.

At this time if not complete the reaction mixture was filtered to remove the catalyst and re-charged to the reaction vessel with a fresh portion of catalyst and the reaction continued for a further 1 hr, the reaction was again sampled for in-process analysis. Celite was charged to a fritted funnel to a height to allow filtration of the catalyst. Once the reaction was shown to be complete the contents of the reaction vessel were filtered, under suction, through the celite into a 5 L filter flask. The reaction vessel was rinsed with methanol (0.74 L) and the washing was filtered through the celite cake. The organic filtrates were combined and then poured into water (5.75 L). The methanol was removed under vacuum on a rotary evaporator (temp 30 to 50° C.) for 2 hr. Following an in-process check to confirm the methanol content of the resulting solution was in the range 5-10% w/w the remaining solution was charged to a 20 L flask and water (8.05 L) was added. The pH of the solution was checked and adjusted if necessary (pH 2.0 to 2.5 was acceptable). The purpose of the pH adjustment is to ensure that the primary amino group in the produce is protonated such that the product will remain in the aqueous phase during the extraction process that follows.

Ethyl acetate (2.3 L) was charged to the solution and the mixture was vigorously agitated for 5 minutes. The two layers were separated and the lower aqueous layer was recharged to the separating funnel. Fresh ethyl acetate (2.3 L) was charged to the solution and the mixture was vigorously agitated for 5 minutes. The two layers were separated and the aqueous transferred to a 20 L reaction vessel. A solution of freshly prepared 2.5M aqueous sodium hydroxide solution was charged to the aqueous layer to adjust the pH of the solution (pH 4.5 to 5.0 was acceptable). The solution was cooled to 0 to 10° C. for 14 to 20 hours followed by filtration of the resulting precipitate, under suction, through a fritted funnel. The reaction vessel was rinsed with water and the washing was filtered through the fritted funnel. The resulting damp solid was transferred to a drying tray and dried in a vacuum oven at 40 to 50° C. for 4 hours, or until constant weight was obtained, to afford cholyl-L-lysine as a white solid.

m/z=537.6 (M+1)

m.p.=216-219° C.

IR (KBr) 3261, 2922, 2854, 1619, 1569, 1461, 1378 cm⁻¹

1. A process for the preparation of cholyl-L-lysine comprising the steps of: —
(a) reacting N-ε-CBZ-cholyl-L-lysine with a hydrogen source in the presence of a catalyst in a solvent comprising one or more alkanols;
(b) removing the catalyst;
(c) optionally diluting the resulting reaction mixture with water and optionally adjusting the pH of the resultant reaction mixture to a pH less than or equal to about 4;
(d) removing the bulk of the alkanol whilst ensuring that the alkanol content of the resultant mixture is maintained above about 3% w/w of the remaining mixture;
(e) extracting the resultant mixture from step (d) with an organic solvent;
(f) adjusting the pH of the aqueous layer to a pH of about 4.5 or greater to precipitate cholyl-L-lysine;
(g) isolate the precipitate.

2. The process according to claim 1, wherein the alkanol is methanol.

3. The process according to claim 1 wherein the solvent in step (a) is a mixture of ethanol and methanol.

4. The process according to claim 3 wherein the ratio of methanol to ethanol is in the range 5 to 1 v/v to 20:1 v/v.

5. The process according to claim 4 wherein the ratio of methanol to ethanol is in the order of 10:1 v/v.

6. The process according to any one of claims 1 to 5 inclusive wherein the alkanol content of the resultant mixture in step (d) is maintained at or above about 5% w/w of the remaining mixture.

7. The process according to claim 6 wherein the alkanol content of the resultant mixture in step (d) is maintained in the range of about 5-10% w/w of the remaining mixture.

8. The process according to claim 1 wherein the hydrogenation process in step (a) is a transfer hydrogenation process.

9. The process according to claim 8 wherein the transfer hydrogenation process utilizes formic acid in methanol.

10. The process according to claim 9 wherein the transfer hydrogenation process is carried out at about 25° C. to 45° C.

11. The process according to claim 10 wherein the transfer hydrogenation process in step (a) is carried out at about 30° C. to 45° C.

12. A The process according to claim 10 wherein the transfer hydrogenation process in step (a) is carried out at 35° C. to 45° C.

13. The process according to any one of claims 8 to 12 inclusive wherein, in the event that the transfer hydrogenation process is incomplete, the catalyst is removed and fresh catalyst is added until complete conversion is observed, re-dosing with catalyst as necessary.

14. The process according to claim 1 wherein the organic solvent used in the extraction process in step (e) is ethyl acetate.

15. (canceled)
